(19) United States
(12) Patent Application Publication
(10) Pub. No.: US 2007/0087355 A1
(43) Pub. Date: Apr. 19, 2007

(54) Comparative genomic hybridization assays and compositions for practicing the same

(76) Inventor: Michael T. Barrett, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/251,048

(22) Filed: Oct. 14, 2005

(51) Int. Cl. C12Q 1/68; C12P 19/34

(52) U.S. Cl. 435/6; 435/91.2

(57) ABSTRACT

Comparative genomic hybridization (CGH) assays and compositions for use in practicing the same are provided. Aspects of the methods include first preparing genomic templates from an initial genomic source by using precursors of circular template nucleic acids, e.g., padlock probes. The precursors include first and second domains that are at least partially complementary to substantially neighboring regions of a genomic domain of interest. In certain embodiments, the methods include an isothermal amplification step, e.g., a rolling circle amplification step. The resultant templates may then be employed to produce target nucleic acid populations, e.g., for use in CGH applications. Also provided are kits for use in practicing the subject methods.

### BACKGROUND OF THE INVENTION

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to neoplastic transformation and subsequent progression can facilitate efforts to define the biological basis for disease, improve prognostication of therapeutic response, and permit earlier tumor detection. In addition, perinatal genetic problems frequently result from loss or gain of chromosome segments such as trisomy 21 or the micro deletion syndromes. Thus, methods of perinatal detection of such abnormalities can be helpful in early diagnosis of disease.

Comparative genomic hybridization (CGH) is one approach that has been employed to detect the presence and identify the location of amplified or deleted sequences. In one implementation of CGH, genomic DNA is isolated from normal reference cells, as well as from test cells (e.g., tumor cells). The two nucleic acids are differentially labeled and then simultaneously hybridized in situ to metaphase chromosomes of a reference cell. Chromosomal regions in the test cells which are at increased or decreased copy number can be identified by detecting regions where the ratio of signal from the two DNAs is altered. For example, those regions that have been decreased in copy number in the test cells will show relatively lower signal from the test DNA than the reference compared to other regions of the genome. Regions that have been increased in copy number in the test cells will show relatively higher signal from the test DNA.

In a recent variation of the above traditional CGH approach, the immobilized chromosome element has been replaced with a collection of solid support surface-bound polynucleotides, e.g., an array of surface-bound BAC, cDNA or oligonucleotide probes for regions of a genome. Such approaches offer benefits over immobilized chromosome approaches, including a higher resolution, as defined by the ability of the assay to localize chromosomal alterations to specific areas of the genome.

In certain applications, archival tissue samples represent an invaluable resource for both diagnostic and prognostic determinations, as well as the ability to correlate disease states with genetic disorders, including single nucleotide polymorphisms (SNPs), aberrant gene expression, chromosomal and gene rearrangement, translocation and/or alternate splicing, and chromosomal duplication/elimination. However, using archived samples, such as formalin-fixed, paraffin-embedded and/or ethanol-fixed samples presents a number of problems generally associated with nucleic acid degradation and variability. See Karsten et al., Nucleic Acids Research Vol. 30, No. 2 e4, pages 1-9, expressly incorporated herein by reference. For example, a degraded genomic sample may have to be reconstructed to produce a suitable genomic template from which probe molecules adequate for use in CGH may be employed.

There is continued interest in the development of improved array-based CGH methods. Of particular interest would be the development of improved array based CGH methods in which archived (or similarly degraded) samples may be assayed.

Relevant Literature

Published of interest include: U.S. Pat. Nos: 6,465,182; 6,355,431; 6,335,167; 6,251,601; 6,210,878; 6,197,501; 6,159,685; 5,965,362; 5,830,645; 5,665,549; 5,447,841 and 5,348,855, as well as published U.S. Application Serial Nos. 2002/0006622; 2004/0241658; 2004/0191813 and 2004/0259105; and published PCT application WO 95/22623. Articles of interest include: Landegren et al.,“Molecular tools for a molecular medicine: analyzing genes, transcripts and proteins using padlock and proximity probes,” J. Mol. Recognit. (2004) 17(3):194-7; Baner et al., “Parallel gene analysis with allele-specific padlock probes and tag microarrays,” Nucleic Acids Res. (2003) 31(17):e103; Nilsson et al., “Making ends meet in genetic analysis using padlock probes,” Hum Mutat. (2002)19(4):410-5; Baner et al., “More keys to padlock probes: mechanisms for high-throughput nucleic acid analysis, “Curr. Opin. Biotechnol. (2001)12(1):11-5; and Baner et al., “Signal amplification of padlock probes by rolling circle replication, Nucleic Acids Res. (1998)15;26(22):5073-8.

### SUMMARY OF THE INVENTION

Comparative genomic hybridization (CGH) assays and compositions for use in practicing the same are provided. Aspects of the methods include first preparing genomic templates from an initial genomic source by using precursors of circular template nucleic acids, e.g., padlock probes. The precursors include first and second domains that are at least partially complementary to substantially neighboring regions of a genomic domain of interest. In certain embodiments, the methods include an isothermal amplification step, e.g., a rolling circle amplification step. The resultant templates may then be employed to produce target nucleic acid populations, e.g., for use in CGH applications. Also provided are kits for use in practicing the subject methods.

### BRIEF DESCRIPTION OF THE FIGURES

FIG. 1 shows a schematic diagram of a method according to a representative embodiment of the invention.

### DEFINITIONS

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length, or longer, e.g., up to about 500 nucleotides or longer. Oligonucleotides are usually synthetic and, in certain embodiments, are under 100, e.g., under 50 nucleotides in length.

The term “oligomer” is used herein to indicate a chemical entity that contains a plurality of monomers. As used herein, the terms “oligomer” and “polymer” are used interchangeably, as it is generally, although not necessarily, and includes smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like:

The phrase “surface-bound polynucleotide” refers to a polynucleotide that is immobilized on a surface of a solid substrate, where the substrate can have a variety of configurations, e.g., a sheet, bead, or other structure. In certain embodiments, the collections of oligonucleotide target elements employed herein are present on a surface of the same planar support, e.g., in the form of an array.

The phrase “labeled population of nucleic acids” refers to a mixture(s) of nucleic acids that are detectably labeled, e.g., fluorescently labeled, such that the presence of the nucleic acids can be detected by assessing the presence of the label.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100 μ², or even smaller. For example, features may have widths (that is, diameter, for a round spot) in the range from 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and is less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular sequence. Array features are typically, but need not be, separated by intervening spaces. In the case of an array in the context of the present application, the “population of labeled nucleic acids” will be referenced as a moiety in a mobile phase (typically fluid), to be detected by “surface-bound polynucleotides” which are bound to the substrate at the various regions. These phrases are synonymous with the terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as signals (e.g., electrical, optical, radio signals, etc.) over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C. and washing in 0.1×SSC/0.1% SDS at 68°°C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “pre-determined” refers to an element whose identity or composition is known prior to its use. For example, a “pre-determined chromosome composition” is a composition containing chromosomes of known identity. An element may be known by name, sequence, molecular weight, its function, or any other attribute or identifier.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface bound polynucleotides, as is commonly known in the art and described below, is not a mixture of capture agents because the species of surface bound polynucleotides are spatially distinct and the array is addressable. “Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well known in the art and include, for example, ion-exchange chromatography, affinity chromatography, flow sorting, and sedimentation according to density.

The term “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional application, and, as such, means employing, e.g. putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

“Contacting” means to bring or put together. As such, a first item is contacted with a second item when the two items are brought or put together, e.g., by touching them to each other.

A “probe” means a polynucleotide which can specifically hybridize to a target nucleotide, either in solution or as a surface-bound polynucleotide. In 25 the case of an array in the context of the present application, the “target” may be referenced as a moiety in a mobile phase (typically fluid), to be detected by “probes” which are bound to the substrate at the various regions.

The term “validated probe” means a probe that has passed at least one screening or filtering process in which experimental data related to the performance of the probes was used as part of the selection criteria.

“In silico” means those parameters that can be determined without the need to perform any experiments, by using information either calculated de novo or available from public or private databases.

The term “genome” refers to all nucleic acid sequences (coding and non-coding) and elements present in or originating from any virus, single cell (prokaryote and eukaryote) or each cell type and their organelles (e.g. mitochondria) in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

By “genomic source” is meant the initial nucleic acids that are used as the original nucleic acid source from which the solution phase nucleic acids are produced, e.g., as a template in the labeled solution phase nucleic acid generation protocols described in greater detail below.

The genomic source may be prepared using any convenient protocol. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. However, in certain embodiments, the genomic source may comprise a portion of the genome, e.g., one or more specific chromosomes or regions thereof, such as PCR amplified regions produced with a pairs of specific primers.

A given initial genomic source may be prepared from a subject, for example a plant or an animal, which subject is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 Mb, such that they may be about 1 Mb or smaller, e.g., less than about 500 Kb, etc.

In certain embodiments, the genomic source is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in certain embodiments are human or mouse genomic sources. In certain embodiments, a set of nucleic acid sequences within the genomic source is complex, as the genome contains at least about 1×10⁸ base pairs, including at least about 1×10⁹ base pairs, e.g., about 3×10⁹ base pairs.

Where desired, the initial genomic source may be fragmented in the generation protocol, as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc.

Where desired, the initial genomic source may be amplified as part of the solution phase nucleic acid generation protocol, where the amplification may or may not occur prior to any fragmentation step. In those embodiments where the produced collection of nucleic acids has substantially the same complexity as the initial genomic source from which it is prepared, the amplification step employed is one that does not reduce the complexity, e.g., one that employs a set of random primers, as described below. For example, the initial genomic source may first be amplified in a manner that results in an amplified version of virtually the whole genome, if not the whole genome, before labeling, where the fragmentation, if employed, may be performed pre-or post-amplification.

The term “amplification” refers to the process in which “replication” is repeated in cyclic process such that the number of copies of the nucleic acid sequence is increased in either a linear or logarithmic fashion. Such replication processes may include but are not limited to, for example, Polymerase Chain Reaction (PCR), Rolling Circle Amplification (RCA), etc.

The term “ligase” refers to an enzyme that catalyzes the formation of a phosphodiester bond between adjacent 3′ hydroxyl and 5′ phosphoryl termini of oligonucleotides that are hydrogen bonded to a complementary strand and the reaction is termed “ligation.”

The term “ligation” refers to joining of 3′ and 5′ ends of two proximal positioned nucleic acids, e.g., DNAs, such as 3′ and 5′ ends of a precursor molecule of the invention, by an enzyme having nucleic acid having ligase activity.

### DESCRIPTION OF THE SPECIFIC EMBODIMENTS

Comparative genomic hybridization (CGH) assays and compositions for use in practicing the same are provided. Aspects of the methods include first preparing genomic templates from an initial genomic source by using precursors of circular template nucleic acids, e.g., padlock probes. The precursors include first and second domains that are at least partially complementary to substantially neighboring regions of a genomic domain of interest. In certain embodiments, the methods include an isothermal amplification step, e.g., a rolling circle amplification step. The resultant templates may then be employed to produce target nucleic acid populations, e.g., for use in CGH applications. Also provided are kits for use in practicing the subject methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the present invention provides methods for comparing populations of nucleic acids and compositions for use therein, where the invention is particularly suited for use with initial archived nucleic acid sample amounts. In further describing the present invention, the subject methods are discussed first in greater detail, followed by a review of representative kits for use in practicing the subject methods.

METHODS

Aspects of the subject invention provide methods for comparing populations of nucleic acids and compositions for use therein, where a feature of the subject methods is the use of genomic templates prepared from initial genomic sources using precursors of circular templates, e.g., padlock probes, that are specific for genomic regions of interest.

In practicing representative embodiments of the subject methods, one generates at least two different populations or collections of target nucleic acids from two or more genomic templates, where the genomic templates are prepared as described below. The two or more populations of target nucleic acids may or may not be labeled, depending on the particular detection protocol employed in a given assay. For example, in certain embodiments, binding events on the surface of a substrate may be detected by means other than by detection of a labeled probe nucleic acids, such as by change in conformation of a conformationally labeled immobilized target, detection of electrical signals caused by binding events on the substrate surface, etc. In certain embodiments, however, the populations of target nucleic acids are labeled, where the populations may be labeled with the same label or different labels, depending on the actual assay protocol employed. For example, where each population is to be contacted with different but identical probe arrays, each target nucleic acid population or collection may be labeled with the same label. Alternatively, where both populations are to be simultaneously contacted with a single array of probes, i.e., cohybridized to the same array of immobilized probe nucleic acids, the populations are generally distinguishably or differentially labeled with respect to each other.

The two or more (i.e., at least first and second, where the number of different collections may, in certain embodiments, be three, four or more) populations of target nucleic acids are prepared from different genomic templates that are, in turn, prepared from different genomic sources.

As such, the first step in many embodiments of the subject methods is to prepare a genomic template from an initial genomic source for each genome that is to be compared. The next step in many embodiments of the subject methods is to then prepare a collection of target nucleic acids, e.g., labeled target nucleic acids, from the prepared genomic template for each genome that is to be compared. Each of these initial steps is now described separately in greater detail.

While in the broadest sense any genomic source may be employed as the initial starting material, in certain embodiments, the initial genomic source is one that is an archived genomic source. By “archived genomic source” is meant a source of nucleic acids obtained from archived tissue samples, particularly paraffin and polymer embedded samples, ethanol embedded samples and/or formalin and formaldehyde embedded tissues. Archived genomic sources may be characterized by the presence of nucleic acid degradation, variability and generally poor condition of such samples. A feature of the archived genomic-sources is that the genomic material may be degraded, such that the average molecular length of the polynucleotides making up the genomic source ranges from about 10 nt to about 10,000 nt, such as from about 25 nt to about 5,000 nt, including from about 50 nt to about 500 nt. Nucleic acids isolated from these samples can be highly degraded and the quality of nucleic preparation can depend on several factors, including the sample shelf life, fixation technique and isolation method. However, using the methodologies outlined herein, highly reproducible results can be obtained that closely mimic results found in fresh samples.

Following obtainment of the initial genomic source, the initial genomic source is contacted with one or more, including a plurality of, target specific precursors of a circular template nucleic acid, e.g., a padlock probe. A target specific precursor of a circular template nucleic acid is a linear nucleic acid molecule that includes a target sequence that is substantially complementary to a genomic region of interest, e.g., a genomic region present in a probe molecule on a CGH array. The target sequence is typically apportioned or present in two separate domains of the precursor molecule, e.g., at least a first domain and a second domain. The target sequence may be evenly or unevenly distributed or apportioned among these two domains. The first and second domains are generally located at opposite ends of the precursor molecule and are sufficiently complementary to substantially neighboring regions of a target genomic domain or region.

By sufficiently complementary is meant that that, under stringent conditions, the first and second domains simultaneously hybridize to the target genomic domain to which they have complementarity. The first and second domains hybridize to substantially neighboring regions of the genomic target domain such that, under appropriate conditions, they may be joined together via a genomic target domain mediated ligation event to produce a circular nucleic acid. Two regions are considered substantially neighboring if the distance of the genomic domain that is not hybridized to a nucleic acid between first and second domains does not exceed about 5 nt, such as 4 nt, such as 3 nt, such as 2 nt, such as 1 nt, such as 0 nt. In certain embodiments, the distance is determined when a third linker nucleic acid is employed in connection with the precursor, e.g., as reviewed in WO 95/22623.

The overall length of a precursor nucleic acid employed in the subject methods may vary, but in representative embodiments may range from about 50 to about 500 nt or longer, e.g., from about 75 to about 250 nt, such as from about 100 to about 175 nt. Each of the first and second domains may range in length from about 10 to about 100 nt, such as from about 20 to about 50 nt, e.g., from about 25 or 30 to about 40 nt. The complementarity between a first or second domain and its corresponding region of the target genome for which the precursor has been designed may be at least about 75%, such as at least about 80%, including at least about 90%, 95%, 99% or more (e.g., as determined using the BLAST algorithm with default settings).

In certain embodiments, the subject precursor nucleic acids include a third domain separating the first and second domains. In certain embodiments, the third domain that separates the first and second domains contains a restriction endonuclease site. The length of the third domain may vary, and in representative embodiments ranges from about 4 to about 500 nt, such as from about 10 to about 300 nt, including from about 20 to about 100 nt.

As mentioned above, in certain embodiments, the third domain includes at least one restriction endonuclease recognized site, i.e. restriction endonuclease site or restriction site, e.g., which serves as mechanism for cleaving a product nucleic acid, as described in greater detail below. A variety of restriction sites are known in the art and may be included, where such sites include (but are not limited to) those recognized by the following restriction enzymes: HindIII, PstI, SaII, AccI, HincII, XbaI, BamHI, SmaI, XmaI, KpnI, Sacl, EcoRI, and the like.

As reviewed above, aspects of the invention include contacting a genomic source with at least one precursor of a circular template nucleic acid.

In certain embodiments, the genomic source is contacted with a plurality of different or distinct precursors, where each distinct type of precursor in the plurality is specific for a different genomic domain, e.g., where the different genomic domains have the sequences found in different probes or features thereof of a CGH array. By plurality is meant at least 2, such as at least about 5, including at least about 10 different precursors of differing sequence, where the number of distinct precursors of differing sequence in a given plurality may be at least about 25, at least about 50, at least about 100, at least about 500, 30 at least about 1000 or more, such as at least about 5,000 or more, at least about 10,000 or more, at least about 25,000 or more, etc. In certain embodiments, the precursors that are contacted with the genomic source are selected for at least a portion of (e.g., at least about 50, at least about 60, at least about 70, at least about 80, at least about 90 number %), including all of, the probes of a pre-identified CGH array, so that targets that are generated from a genomic source are targets for probes that are found on a pre-identified array to be employed with the generated targets.

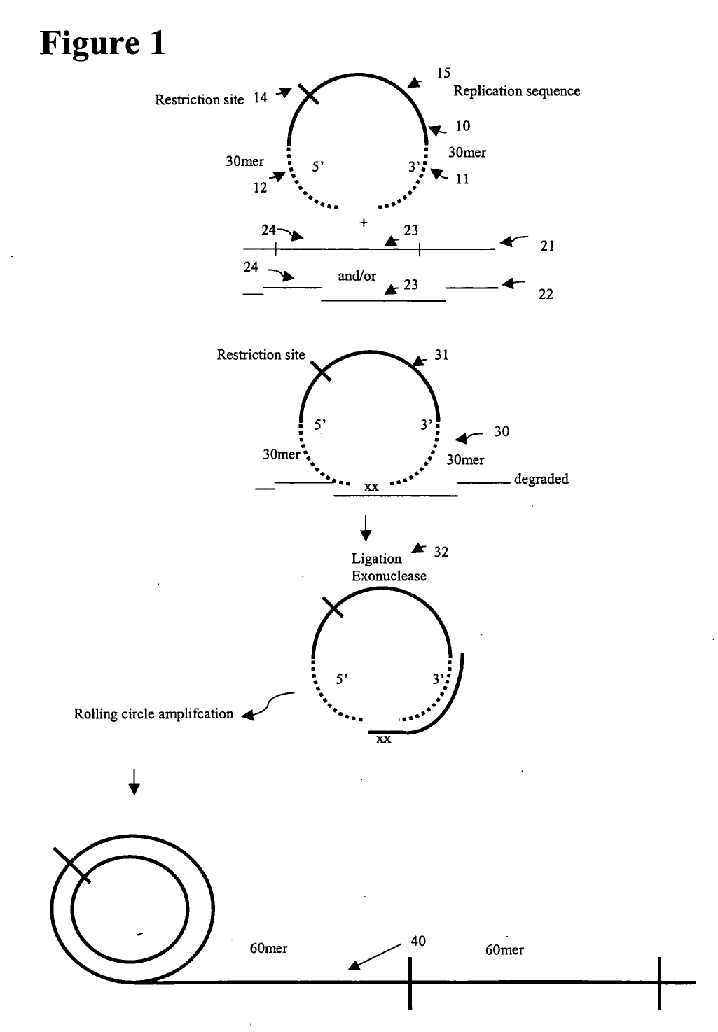

The genomic source and the precursor(s) are contacted in a manner sufficient to generate circular template molecules from the precursors. Specifically, the circular template molecules are produced from the precursors that hybridize to a complementary genomic domain present in the genomic source. As illustrated in FIG. 1, contact of the precursor(s) and the source occurs in a manner that results in the production of circular structures of any precursors and their corresponding genomic domains present in the source, where the entire corresponding domain may be present on a single source molecule, or only a portion of the corresponding domain may be present in the source molecule.

To stabilize the resultant circular structures, the ends of the first and second domains of the circular structures are ligated to each other, e.g., optionally through a linker molecule as described in WO 95/22623, to produce circular template nucleic acids. Specifically, as depicted in FIG. 1, the first and second domains of the circular strand are ligated together in a genomic domain mediated ligation reaction to produce continuous or stabilized circular template nucleic acids.

As such, in representative embodiments contact of the precursor and the genomic source occurs under ligation conditions. In these representative embodiments, ligation of the precursor first and second domains of the precursor which are hybridized to substantially neighboring, if not immediately adjacent, regions of the genomic domain, is achieved by contacting the reaction mixture with a nucleic acid ligating activity, e.g., provided by a suitable nucleic acid ligase, and maintaining the product thereof under conditions sufficient for ligation of the first and second domain to occur.

In representative embodiments of the subject invention, the first and second nucleic acid domains are ligated to each other in this ligation step by using a ligase. As is known in the art, ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary. Any convenient ligase may be employed, where representative ligases of interest include, but are not limited to: temperature sensitive and thermostable ligases. Temperature sensitive ligases, include, but are not limited to, bacteriophage T4 DNA ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eucaryotic, or archael organisms. Certain RNA ligases may also be employed in the methods of the invention.

In this ligation step, a suitable ligase and any reagents that are necessary and/or desirable are combined with the reaction mixture and maintained under conditions sufficient for ligation of the hybridized ligation oligonucleotides to occur. Ligation reaction conditions are well known to those of skill in the art. During ligation, the reaction mixture in certain embodiments may be maintained at a temperature ranging from about 20° C. to about 45° C., such as from about 25° C. to about 37° C. for a period of time ranging from about 5 minutes to about 16 hours, such as from about 1 hour to about 4 hours. In yet other embodiments, the reaction mixture may be maintained at a temperature ranging from about 35° C. to about 45° C., such as from about 37° C. to about 42° C., e.g., at or about 38° C., 39° C., 40° C. or 41° C., for a period of time ranging from about 5 minutes to about 16 hours, such as from about 1 hour to about 10 hours, including from about 2 to about 8 hours. In a representative embodiment, the ligation reaction mixture includes 50 mM Tris pH7.5, 10 mM MgCl₂, 10 mM DTT, 1 mM ATP, 25 mg/ml BSA, 0.25 units/ml Rnase inhibitor, and T4 DNA ligase at 0.125 units/ml. In yet another representative embodiment, 2.125 mM magnesium ion, 0.2 units/ml Rnase inhibitor; and 0.125 units/ml DNA ligase are employed.

In certain embodiments, the reaction mixture produced as described above is subject to one or more cycles of denaturation and re-annealing, e.g., to ensure that only precursors that correctly match up or are hybridized to sequences in the genomic source are converted to circular template molecules. Denaturation and re-annealing may be achieved using any convenient protocol. In one representative embodiment, denaturation and re-annealing is achieved by subjecting the mixture to one or more cycles of heating and cooling. For example, the mixture may be subjected to strand disassociation conditions, e.g., subjected to a temperature ranging from about 80° C. to about 100° C., usually from about 90° C. to about 95° C. for a period of time, e.g., from about 1 to 10 minute, such as from about 1 to 5 minutes, e.g., about 2 minutes, and the resultant disassociated template molecules are then subject to annealing conditions, where the temperature of the composition is reduced, e.g., at a rate of about 0.1° C./sec to about 10° C./sec, to an annealing temperature of from about 20° C. to about 80° C., usually from about 37° C. to about 65° C., and maintained at this temperature for a period of time ranging from about 1 to about 60 minutes. In certain embodiments, a “snap-cooling” protocol is employed, where the temperature is reduced to the annealing temperature, or to about 40° C. or below in a period of from about 1s to about 30s, usually from about 5s to about 10s.

Where two more cycles of heating and cooling are applied to the mixture, the number of cycles may be at least about 5, such as at least about 10, including 15 or more, 20 or more, etc.

The above step of the subject methods results in a product mixture characterized by the presence of circular template molecules, which are continuous circular molecules produced by ligation of the first and second domains of initial precursors. The circular template molecules present in the product mixture are ones that are produced only from precursors that bound to complementary genomic molecules in the genomic source. As such, the circular template molecules present in the product mixture provide an accurate representation of the different genomic sequences of interest present in the genomic source. For example, where a genomic source has two copies of regions 1, 2, 3, 4 and 5 and three copies of region 6 but no copies of region 7, when precursors for regions 1, 2, 3, 4, 5, 6 and 7 are employed as described above, one will obtain approximately equal amounts of circular template nucleic acids for regions 1 through 5, an amount of circular template for region 6 that is approximately 1.5 times the amount obtained for any other region, and no circular templates for region 7.

Where desired, the resultant product mixture of the above steps may be treated to remove any unwanted byproducts, e.g., unligated or mismatched sequences. Treatment may be achieved using any convenient protocol, e.g., by contacting the mixture with an exonuclease. As is known in the art, exonucleases act on the terminal of polynucleotide chain of nucleic acid molecule and hydrolyze the chain progressively to liberate nucleotides. Reviews about nucleases and their applications include: Williams RJ. Methods Mol Biol 2001;160:409-429; Meiss G, Gimadutdinow O, Friedhoff P, Pingoud AM. Methods Mol Biol 2001 ;160:37-48; Fors L, Lieder KW, Vavra SH, Kwiatkowski RW. Pharmacogenomics 2000 May; 1(2):219-229;Cappabianca L, Thomassin H, Pictet R, Grange T. Methods Mol Biol 1999;119: 427-442; Taylor GR, Deeble J. Genet Anal 1999 February;14(5-6):181-186; Suck D. Biopolymers 1997;44(4):405-421; Liu QY, Ribecco M, Pandey S, Walker PR, Sikorska M. Ann N Y Acad Sci 1999;887:60-76; Liao TH. J Formos Med Assoc 1997 July;96(7):481-487; Suck D. J Mol Recognit 1994 June;7(2):65-70; and Liao TH. Mol Cell Biochem 1981January 20;34(1):15-22. Specific exonucleases of interest for this step include, but are not limited to: DNA exonucleases I and III and the like.

The resultant product is characterized by the presence of circular template molecules, and specifically single stranded circular molecules, where the circular template molecules may or may not be partially hybridized to a portion of a genomic sequence, e.g., as depicted in FIG. 1.

The next step of the subject methods is to convert the resultant ligation production mixture, as described above, to a genomic template. Generally, this conversion step includes subjecting the resultant ligation product mixture to template dependent primer extension reaction conditions. This conversion step may include a variety of different specific protocols, where the protocols may or may not include an amplification step, as may be desired.

In one representative conversion protocol, an amplification step is not included. In this representative protocol, the resultant circular template nucleic acid is contacted with a suitable primer, e.g., that hybridizes to a universal priming site, e.g., located in the third domain of the circular template, a polymerase and the appropriate deoxynucleotides (i.e., dGTP, dCTP, dATP and dTTP) and maintained under primer extension conditions such that the a second strand DNA is synthesized under a template dependent primer extension reaction, where the circular template serves as the template strand. As such, this protocol is representative of non-amplification conversion protocols. Primer extension reaction conditions and reagents employed therein, e.g., polymerases, buffers, etc., are well known in the art and need not be described in greater detail here. It should be noted that in the above and below protocols, primer may not be required in certain embodiments, as the genomic sequence hybridized to the template may serve as primer.

In other embodiments, it is desirable to employ a conversion protocol that includes amplification, such that amplified amounts of product linear DNA molecules are produced for an initial circular template. Any convenient amplification conversion protocol may be employed.

One representative amplification conversion protocol of interest is a protocol that employs “rolling circle amplification” or RCA. In these rolling circle amplification protocols, the circular single-stranded template molecule serves as a template for rolling circle amplification (which may be linear or geometric, but is generally linear), in which at least one, if not two, e.g., forward and reverse, rolling circle primer is contacted with the circular template under rolling circle amplification conditions sufficient to produce long nucleic acids that include multiple copies of the desired genomic target domain. Rolling circle amplification conditions are known in the art and described in, among other locations, U.S. Pat. Nos. 6,576,448; 6,287,824; 6,235,502; and 6,221,603; the disclosures of which are herein incorporated by reference.

For rolling circle amplification, the circular template strand is contacted with at least one primer, a suitable polymerase, and the four dNTPs, as well as any other desired reagents to produce a rolling circle amplification reaction mixture, which reaction mixture is then maintained under rolling circle amplification conditions. In certain embodiments, the polymerase that is employed is a highly processive polymerase. By highly processive polymerase is meant a polymerase that elongates a DNA chain without dissociation over extended lengths of nucleic acid, where extended lengths means at least about 50 nt long, such as at least about 100 nt long or longer, including at least about 250 nt long or longer, at least about 500 nt long or longer, at least about 1000 nt long or longer. In many embodiments, the polymerase employed in the amplification step is a phage polymerase. Of interest in certain embodiments is the use of a φ29-type DNA polymerase. By φ29-type DNA polymerase is meant either: (i) that phage polymerase in cells infected with a φ29-type phage; (ii) a φ29-type DNA polymerase chosen from the DNA polymerases of phages φ29, Cp-1, PRD1, f15, f21, PZE, PZA, Nf, M2Y, B103, SF5, GA-1, Cp-5, Cp-7, PR4, PR5, PR722, and L17; or (iii) a φ29-type polymerase modified to have less than ten percent of the exonuclease activity of the naturally-occurring polymerase, e.g., less than one percent, including substantially no, exonuclease activity. Representative φ29 type polymerases of interest include, but are not limited to, those polymerases described in U.S. Pat. No. 5,198,543, the disclosure of which is herein incorporated by reference. This particular embodiment is representative of isothermal amplification embodiments. As such, in certain embodiments, the amplification protocol employed is an isothermal strand displacement protocol. By isothermal is meant that the protocol does not employ thermal cycling.

In yet another representative amplification, the conversion protocol is a polymerase chain reaction (PCR) protocol, in which the circular template molecule is contacted with appropriate primer(s), a suitable polymerase and the appropriate deoxynucleotides to produce a PCR reaction mixture, which PCR reaction mixture is then subjected to polymerase chain reaction (PCR conditions), where the reaction may provide for linear or geometric amplification. The polymerase chain reaction (PCR) is well known in the art, being described in U.S. Pat. Nos.: 4,683,202; 4,683,195; 4,800,159; 4,965,188 and 5,512,462, the disclosures of which are herein incorporated by reference. By polymerase chain reaction conditions is meant the total set of conditions used in a given polymerase chain reaction, e.g. the nature of the polymerase or polymerases, the type of buffer, the presence of ionic species, the presence and relative amounts of dNTPs, etc. Using a suitable PCR protocol, multiple copies of a desired linear DNA molecule that includes a copy of the genomic target domain or sequence of interest may be produced from a single intermediate molecule.

The above described conversion step results in the production of a linear nucleic acid, and specifically DNA, molecule that includes at least one copy of the genomic domain of interest, where the resultant molecules may or may not include more than one copy of the domain of interest linearly arranged on the molecule, e.g., each separated by a third domain, depending on the particular conversion protocol that is employed. For example, in the representative non-amplification conversion protocol, the product linear molecules include a single copy of the target sequence of interest. In contrast, in the representative rolling circle amplification protocol described above, the product molecules include multiple copies of the desired target sequence of interest, where each copy is separated from each other by a domain corresponding to the third domain of the precursor.

Where desired, the product may be subjected to one or more rounds of amplification, e.g., by using additional “padlock probes” for the restriction product. As such, the products of the first RCA may be linearized by restriction digestion, converted to new DNA circles, and then reannealed to padlock probes complementary to sequences in the RCA templates. These latter padlock probes would be of opposite polarity to the first set of padlock probes. This process of linearization, ligation and RCA can be repeated one or more times according to the experimental needs.

A representative embodiment of the above methods is shown schematically in FIG. 1. In the embodiment shown in FIG. 1, the precursor nucleic acid 10 is a padlock probe. In the padlock probe 10, each terminus of the molecule (11, 12) (also referred to as the first and second domains of the probes) contains sequence complementary to the genomic target domain found in either an intact or degraded genomic source, 21 and 22 respectively. That is, the first end 11 of the padlock probe is substantially complementary to a first target domain 23, and the second end of the RCA probe is substantially complementary to a second target domain 24, adjacent to the first domain. Hybridization of the precursor 10 to the target nucleic acid results in the formation of a hybridization complex 30 containing a circular probe, e.g., which, following ligation of the termini, may be employed as an RCA template. That is, the probe is circularized while still hybridized with the target nucleic acid, as shown by step 32. This serves as a circular template for RCA. Addition of a polymerase to the RCA template complex results in the formation of an amplified product nucleic acid 40.

As shown in the embodiment depicted in FIG. 1, the padlock probe 10 contains a restriction site 14 present in a third domain, labeled replication sequence 15. The restriction endonuclease site allows for cleavage of the long concatamers that are typically the result of RCA into smaller individual units, as desired. Thus, following RCA, the product nucleic acid is contacted with the appropriate restriction endonuclease (not shown). This step results in cleavage of the product nucleic acid into smaller fragments. The fragments are then employed as template, as described below.

The padlock probe employed in the embodiment depicted in FIG. 1 typically contains a priming site for priming the RCA reaction. That is, the padlock probe comprises a sequence to which a primer nucleic acid hybridizes forming a template for the polymerase. The primer can be found in any portion of the circular probe, but in representative embodiments is located at a discrete site in the probe, e.g., in the replication sequence or third domain 15. In this embodiment, the primer site in each distinct padlock probe is identical, although this is not required. Advantages of using primer sites with identical sequences include the ability to use only a single primer oligonucleotide to prime the RCA assay with a plurality of different hybridization complexes. That is, the padlock probe hybridizes uniquely to the target nucleic acid to which it is designed. A single primer hybridizes to all of the unique hybridization complexes forming a priming site for the polymerase. RCA then proceeds from an identical locus within each unique padlock probe of the hybridization complexes. In an alternative embodiment, the primer site can overlap, encompass, or reside within any of the above-described elements of the padlock probe. That is, the primer can be found, for example, overlapping or within the restriction site or the identifier sequence.

Where desired, the product of the above steps of the subject methods is further treated prior to its subsequent use, e.g., as genomic template in a CGH application. For example, the product may be purified, as well as quantitated, where numerous representative protocols for such are well known to those of skill in the art.

The above steps result in the production of a genomic template for each initial genomic source. Where the genomic source employed to produce the genomic template is an archived source, a feature of the subject methods is that the product genomic template is comparable with the genomic templates obtained from fresh tissue. In addition, when quantitation is performed, the present methods provide for highly reproducible results between archived samples such that, for example, sets of cancerous versus non-cancerous tissue samples can be compared. In a representative embodiment, the results from archived samples are within 20% of those for fresh samples; such as within 10% of each other and including within 5 or 1% of each other. In addition, when genotyping is performed, the difference between the fresh and archived samples is less than 10%; such as less than 5 or 1% and including than 0.5% in certain embodiments.

Following provision of the genomic template, and any initial processing steps (e.g., fragmentation, etc.) as described above, a collection of target nucleic acids is prepared from the genomic template for use in the subject methods. In certain embodiments of particular interest, the collection of target nucleic acids prepared from the genomic template is one that has substantially the same complexity as the complexity of the genomic template, and in certain embodiments the initial genomic source. See e.g., U.S. patent application Ser. No. 10/744,595 for its discussion of complexity, which is incorporated herein by reference.

In representative embodiments of interest, the collection or population of target nucleic acids that is prepared in this step of the subject methods is one that is labeled with a detectable label. In the embodiments where the population of target nucleic acids is a non-reduced complexity population of nucleic acids, as described in Ser. No. 10/744,595, the labeled target nucleic acids are prepared in a manner that does not reduce the complexity to any significant extent as compared to the initial genomic template. A number of different nucleic acid labeling protocols are known in the art and may be employed to produce a population of labeled probe nucleic acids. The particular protocol may include the use of labeled primers, labeled nucleotides, modified nucleotides that can be conjugated with different dyes, one or more amplification steps, etc.

In one type of representative labeling protocol of interest, the genomic template is employed in the preparation of labeled nucleic acids, e.g., as a genomic template from which the labeled nucleic acids are enzymatically produced. Different types of template dependent labeled nucleic acid generation protocols are known in the art. In certain types of protocols, the template is employed in a non-amplifying primer extension nucleic acid generation protocol. In yet other embodiments, the template is employed in an amplifying primer extension protocol.

Of interest in the embodiments described above, whether they be amplifying or non-amplifying primer extension reactions, is the use of a set of primers that results in the production of the desired target nucleic acid collection of high complexity, i.e., comparable or substantially similar complexity to the initial genomic source. In many embodiments, the above described population of target nucleic acids in which substantially all, if not all, of the sequences found in the initial genomic template are present, is produced using a primer mixture of random primers, i.e., primers of random sequence. The primers employed in the subject methods may vary in length, and in many embodiments range in length from about 3 to about 25 nt, sometimes from about 5 to about 20 nt and sometimes from about 5 to about 10 nt. The total number of random primers of different sequence that is present in a given population of random primers may vary, and depends on the length of the primers in the set. As such, in the sets of random primers, which include all possible variations, the total number of primers n in the set of primers that is employed is 4^{Y}, where Y is the length of the primers. Thus, where the primer set is made up of 3-mers, Y=3 and the total number n of random primers in the set is 4³ or 64. Likewise, where the primer set is made up of 8-mers, Y=8 and the total number n of random primers in the set is 4⁸ or 65,536. Typically, an excess of random primers is employed, such that in a given primer set employed in the subject invention, multiple copies of each different random primer sequence is present, and the total number of primer molecules in the set far exceeds the total number of distinct primer sequences, where the total number may range from about 1.0×10¹ to about 1.0×10²⁰, such as from about 1.0×10¹³to about 1.0×10¹⁷, e.g., 3.7×10¹⁵. The primers described above and throughout this specification may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066.

As indicated above, in generating labeled target nucleic acids according to these embodiments of subject methods, the above-described genomic template and random primer population are employed together in a primer extension reaction that produces the desired labeled target nucleic acids. Primer extension reactions for generating labeled nucleic acids are well known to those of skill in the art, and any convenient protocol may be employed, so long as the above described genomic source (being used as a template) and population of random primers are employed. In this step of the subject methods, the primer is contacted with the template under conditions sufficient to extend the primer and produce a primer extension product, either in an amplifying or in a non-amplifying manner (where a non-amplifying manner is one in which essentially a single product is produced per template strand). As such, the above primers are contacted with the genomic template in the presence of a sufficient DNA polymerase under primer extension conditions sufficient to produce the desired primer extension molecules. DNA polymerases of interest include, but are not limited to, polymerases derived from E. coli, thermophilic bacteria, archaebacteria, phage, yeasts, Neurosporas, Drosophilas, primates and rodents. The DNA polymerase extends the primer according to the genomic template to which it is hybridized in the presence of additional reagents which may include, but are not limited to: dNTPs; monovalent and divalent cations, e.g. KCl, MgCl₂; sulfhydryl reagents, e.g. dithiothreitol; and buffering agents, e.g. Tris-Cl.

Extension products that are produced as described above are typically labeled in the present methods. As such, the reagents employed in the subject primer extension reactions typically include a labeling reagent, where the labeling reagent may be the primer or a labeled nucleotide, which may be labeled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g., dCTP. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

In the primer extension reactions employed in the subject methods of these embodiments, the genomic template is typically first subjected to strand disassociation condition, e.g., subjected to a temperature ranging from about 80° C. to about 100° C., usually from about 90° C. to about 95° C. for a period of time, and the resultant disassociated template molecules are then contacted with the primer molecules under annealing conditions, where the temperature of the template and primer composition is reduced to an annealing temperature of from about 20° C. to about 80° C., usually from about 37° C. to about 65° C. In certain embodiments, a “snap-cooling” protocol is employed, where the temperature is reduced to the annealing temperature, or to about 4° C. or below in a period of from about 1s to about 30s, usually from about 5s to about 10s.

The resultant annealed primer/template hybrids are then maintained in a reaction mixture that includes the above-discussed reagents at a sufficient temperature and for a sufficient period of time to produce the desired labeled target nucleic acids. Typically, this incubation temperature ranges from about 20° C. to about 75° C., usually from about 37° C. to about 65° C. The incubation time typically ranges from about 5 min to about 18 hr, usually from about 1 hr to about 12 hr.

In yet other embodiments, the collection of target nucleic acids may be one that is of reduced complexity as compared to the initial genomic source. By reduced complexity is meant that the complexity of the produced collection of target nucleic acids is at least about 20-fold less, such as at least about 25-fold less, at least about 50-fold less, at least about 75-fold less, at least about 90-fold less, at least about 95-fold less, than the complexity of the initial genomic source, in terms of total numbers of sequences found in the produced population of probes as compared to the initial source, up to and including a single gene locus being represented in the collection. The reduced complexity can be achieved in a number of different manners, such as by using gene specific primers in the generation of labeled target nucleic acids, by reducing the complexity of the genomic source used to prepare the probe nucleic acids, etc. As with the above non-reduced-complexity protocols, in these reduced complexity protocols, the target nucleic acids prepared in many embodiments are labeled target nucleic acids. Any convenient labeling protocol, such as the above described representative protocols, may be employed, where the protocols are adapted to provide for the desired reduced complexity, e.g., by using gene specific instead of random primers.

Using the above protocols, at least a first collection of target nucleic acids and a second collection of target nucleic acids are produced from two different genomic templates, e.g., a reference and test genomic template, from two different genomic sources. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a feature of certain embodiments is that the different collections or populations of produced labeled target nucleic acids are all labeled with the same label, such that they are not distinguishably labeled. In yet other embodiments, a feature of the different collections or populations of produced labeled target nucleic acids is that the first and second labels are typically distinguishable from each other. The constituent target members of the above produced collections typically range in length from about 10 to about 10,000 nt, such as from about 25 to about 1000 nt, including from about 50 to about 500 nt.

In the next step of the subject methods, the collections or populations of labeled target nucleic acids produced by the subject methods are contacted to a plurality of probe elements under conditions such that nucleic acid hybridization to the probe elements can occur. The target collections can be contacted to the probe elements either simultaneously or serially. In many embodiments the target compositions are contacted with the plurality of probe elements, e.g., the array of probes, simultaneously. Depending on how the collections or populations are labeled, the collections or populations may be contacted with the same array or different arrays, where when the collections or populations are contacted with different arrays, the different arrays are substantially, if not completely, identical to each other in terms of probe feature content and organization.

A feature of certain embodiments of the present invention is that the substrate immobilized probe nucleic acids are oligonucleotide probe nucleic acids. Probe nucleic acids employed in such applications can be derived from virtually any source. Typically, the probes will be nucleic acid molecules having sequences derived from representative locations along a chromosome of interest, a chromosomal region of interest, an entire genome of interest, a cDNA library, and the like.

The choice of probe nucleic acids to use may be influenced by prior knowledge of the association of a particular chromosome or chromosomal region with certain disease conditions. International Application WO 93/18186 provides a list of chromosomal abnormalities and associated diseases, which are described in the scientific literature. Alternatively, whole genome screening to identify new regions subject to frequent changes in copy number can be performed using the methods of the present invention. In these embodiments, probe elements usually contain nucleic acids representative of locations distributed over the entire genome. In such embodiments, the resolution may vary, where in many embodiments of interest, the resolution is at least about 500 Kb, such as at least about 250 Kb, at least about 200 Kb, at least about 150 Kb, at least about 100 Kb, at least about 50 Kb, including at least about 25 Kb, at least about 10 Kb or higher. By resolution is meant the spacing on the genome between sequences found in the targets. In some embodiments (e.g., using a large number of target elements of high complexity) all sequences in the genome can be present in the array. The spacing between different locations of the genome that are represented in the targets of the collection of targets may also vary, and may be uniform, such that the spacing is substantially the same, if not the same, between sampled regions, or non-uniform, as desired.

In some embodiments, previously identified regions from a particular chromosomal region of interest are used as probes. Such regions are becoming available as a result of rapid progress of the worldwide initiative in genomics. In certain embodiments, the array can include probes which “tile” a particular region (which have been identified in a previous assay), by which is meant that the probes correspond to region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled arrays tailored to the initially identified region to further assay the region at a higher resolution, e.g., in an iterative protocol.

Of interest are both coding and non-coding genomic regions, where by coding region is meant a region of one or more exons that is transcribed into an mRNA product and from there translated into a protein produce, while by non-coding region is meant any sequences outside of the exon regions, where such regions may include regulatory sequences, e.g., promoters, enhancers, introns, etc. In certain embodiments, one can have at least some of the probes directed to non-coding regions and others directed to coding regions. In certain embodiments, one can have all of the probes directed to non-coding sequences. In certain embodiments, one can have all of the probes directed to coding sequences.

The oligonucleotide probes employed in the subject methods are immobilized on a solid support. Many methods for immobilizing nucleic acids on a variety of solid support surfaces are known in the art. For instance, the solid support may be a membrane, glass, plastic, or a bead. The desired component may be covalently bound or noncovalently attached through nonspecific binding, adsorption, physisorption or chemisorption. The immobilization of nucleic acids on solid support surfaces is discussed more fully below.

A wide variety of organic and inorganic polymers, as well as other materials, both natural and synthetic, may be employed as the material for the solid surface. Illustrative solid surfaces include nitrocellulose, nylon, glass, fused silica, diazotized membranes (paper or nylon), silicones, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition substances that form gels can be used. Such materials include proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and enhance signal detection or the like.

If covalent bonding between a compound and the surface is desired, the surface will usually include appropriate functionalities to provide for the covalent attachment. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces are well known and is amply illustrated in the literature. For example, methods for immobilizing nucleic acids by introduction of various functional groups to the molecules are known (see, e.g., Bischoff et al., Anal. Biochem. 164:336-344 (1987); Kremsky et al., Nuc. Acids Res. 15:2891-2910 (1987)). Modified nucleotides can be placed on the target using PCR primers containing the modified nucleotide, or by enzymatic end labeling with modified nucleotides, or by non-enzymatic synthetic methods

Use of membrane supports (e.g., nitrocellulose, nylon, polypropylene) for the nucleic acid arrays of the invention is advantageous in certain embodiments because of well-developed technology employing manual and robotic methods of arraying targets at relatively high element densities (e.g., up to 30-40/cm²). In addition, such membranes are generally available and protocols and equipment for hybridization to membranes is well known. Many membrane materials, however, have considerable fluorescence emission, where fluorescent labels are used to detect hybridization.

To optimize a given assay format one of skill can determine sensitivity of fluorescence detection for different combinations of membrane type, fluorochrome, excitation and emission bands, spot size and the like. In addition, low fluorescence background membranes have been described (see, e.g., Chu et al., Electrophoresis 13:105-114 (1992)).

The sensitivity for detection of spots of various diameters on the candidate membranes can be readily determined by, for example, spotting a dilution series of fluorescently end labeled DNA fragments. These spots are then imaged using conventional fluorescence microscopy. The sensitivity, linearity, and dynamic range achievable from the various combinations of fluorochrome and membranes can thus be determined. Serial dilutions of pairs of fluorochrome in known relative proportions can also be analyzed to determine the accuracy with which fluorescence ratio measurements reflect actual fluorochrome ratios over the dynamic range permitted by the detectors and membrane fluorescence.

Arrays on substrates with much lower fluorescence than membranes, such as glass, quartz, or small beads, can achieve much better sensitivity. For example, elements of various sizes, ranging from the about 1 mm diameter down to about 1 μm can be used with these materials. Small array members containing small amounts of concentrated target DNA are conveniently used for high complexity comparative hybridizations since the total amount of probe available for binding to each element will be limited. Thus it may be advantageous in certain embodiments to have small array members that contain a small amount of concentrated target DNA so that the signal that is obtained is highly localized and bright. Such small array members are typically used in arrays with densities greater than 10⁴ elements/cm². Relatively simple approaches capable of quantitative fluorescent imaging of 1 cm² areas have been described that permit acquisition of data from a large number of members in a single image (see, e.g., Wittrup et. al. Cytometry 16:206-213 (1994)).

Covalent attachment of the probe nucleic acids to glass or synthetic fused silica can be accomplished according to a number of known techniques. Such substrates provide a very low fluorescence substrate, and a highly efficient hybridization environment.

There are many possible approaches to coupling nucleic acids to glass that employ commercially available reagents. For instance, materials for preparation of silanized glass with a number of functional groups are commercially available or can be prepared using standard techniques. Alternatively, quartz cover slips, which have at least 10-fold lower auto fluorescence than glass, can be silanized. In certain embodiments of interest, silanization of the surface is accomplished using the protocols described in U.S. Pat. No. 6,444,268, the disclosure of which is herein incorporated by reference, where the resultant surfaces have low surface energy that results from the use of a mixture of passive and functionalized silanization moieties to modify the glass surface, i.e., they have low surface energy silanized surfaces. Additional linking protocols of interest include, but are not limited to: polylysine as well as those disclosed in U.S. Pat. No. 6,319,674, the disclosure of which is herein incorporated by reference. The probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith et al. Science, 258:1122-1126 (1992)). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

In the subject methods (as summarized above), the copy number of particular nucleic acid sequences in two target collections are compared by hybridizing the targets to one or more probe nucleic acid arrays, as described above. The hybridization signal intensity, and the ratio of intensities, produced by the targets on each of the probe elements is determined. Since signal intensities on a probe element can be influenced by factors other than the copy number of a target in solution, for certain embodiments an analysis is conducted where two labeled populations are present with distinct labels. Thus comparison of the signal intensities for a specific probe element permits a direct comparison of copy number for a given sequence. Different probe elements will reflect the copy numbers for different sequences in the target populations. The comparison can reveal situations where each sample includes a certain number of copies of a sequence of interest, but the numbers of copies in each sample are different. The comparison can also reveal situations where one sample is devoid of any copies of the sequence of interest, and the other sample includes one or more copies of the sequence of interest.

Standard hybridization techniques (using high stringency hybridization conditions) are used. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a description of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos: 6,335,167; 6,197,501; 5,830,645; and 5,665,549; the disclosures of which are herein incorporated by reference.

Generally, nucleic acid hybridizations comprise the following major steps: (1) immobilization of probe nucleic acids; (2) pre-hybridization treatment to increase accessibility of target DNA, and to reduce nonspecific binding; (3) hybridization of the mixture of nucleic acids to the nucleic acid on the solid surface, typically under high stringency conditions; (4) post-hybridization washes to remove nucleic acid fragments not bound in the hybridization and (5) detection of the hybridized nucleic acid fragments. The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between immobilized targets and complementary probes in a sample. Representative high stringency assay conditions that may be employed in these embodiments are provided above.

The above hybridization step may include agitation of the immobilized targets and the sample of probe nucleic acids, where the agitation may be accomplished using any convenient protocol, e.g., shaking, rotating, spinning, and the like.

Following hybridization, the surface of immobilized targets is typically washed to remove unbound probe nucleic acids. Washing may be performed using any convenient washing protocol, where the washing conditions are typically stringent, as described above.

Following hybridization and washing, as described above, the hybridization of the labeled nucleic acids to the probes is then detected using standard techniques so that the surface of immobilized targets, e.g., array, is read. Reading of the resultant hybridized array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, CA. Other suitable devices and methods are described in U.S. patent applications: Ser. No. 09/846125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

Results from the reading or evaluating may be raw results (such as fluorescence intensity readings for each feature in one or more color channels) or may be processed results, such as obtained by subtracting a background measurement, or by rejecting a reading for a feature which is below a predetermined threshold and/or forming conclusions based on the pattern read from the array (such as whether or not a particular target sequence may have been present in the sample, or whether or not a pattern indicates a particular condition of an organism from which the sample came).

In certain embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Utility

The above-described methods find use in any application in which one wishes to compare the copy number of nucleic acid sequences found in two or more populations. One type of representative application in which the subject methods find use is the quantitative comparison of copy number of one nucleic acid sequence in a first collection of nucleic acid molecules relative to the copy number of the same sequence in a second collection.

As such, the present invention may be used in methods of comparing abnormal nucleic acid copy number and mapping of chromosomal abnormalities associated with disease. In many embodiments, the subject methods are employed in applications that use target nucleic acids immobilized on a solid support, to which differentially labeled probe nucleic acids produced as described above are hybridized. Analysis of processed results of the described hybridization experiments provides information about the relative copy number of nucleic acid domains, e.g. genes, in genomes.

Such applications compare the copy numbers of sequences capable of binding to the target elements. Variations in copy number detectable by the methods of the invention may arise in different ways. For example, copy number may be altered as a result of amplification or deletion of a chromosomal region, e.g. as commonly occurs in cancer. Representative applications in which the subject methods find use are further described in U.S. Pat. Nos. 6,335,167; 6,197,501; 5,830,645; and 5,665,549; the disclosures of which are herein incorporated by reference.

The subject methods find particular use in high resolution CGH applications where initially small sample volumes are to be analyzed, such as the small sample volumes described above. Small samples may be derived after purification of subpopulations of cells of interest from a starting tissue sample. For example, single and multi-parameter flow cytometry can identify small numbers of abnormal cells in a background of large numbers of normal cells in a biopsy or mixed cell population. Another technique that may be used to produce small samples of purified cells is laser capture microdissection.

Kits

Also provided are kits for use in the subject invention, where such kits may comprise containers, each with one or more of the various reagents/compositions utilized in the methods, where such reagents/compositions typically at least include: a precursor, e.g., padlock probe, or collection of precursors; and a collection of immobilized oligonucleotide probes, e.g., one or more arrays of oligonucleotide probes (where the precursors correspond to probes on the array, e.g., by sharing commune sequence). Also present may be reagents employed in conversion of circular template to genomic template, e.g., rolling circle amplification reagents, as described above, such as the highly processive polymerases described above. In addition, the kits may include one or more reagents employed in genomic template and/or labeled probe production, e.g., a polymerase, exonuclease resistant primers, random primers, buffers, the appropriate nucleotide triphosphates (e.g. dATP, dCTP, dGTP, dTTP), DNA polymerase, labeling reagents, e.g., labeled nucleotides, and the like. Where the kits are specifically designed for use in CGH applications, the kits may further include labeling reagents for making two or more collections of distinguishably labeled nucleic acids according to the subject methods, an array of target nucleic acids, hybridization solution, etc.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

The following examples are offered by way of illustration and not by way of limitation.

### EXPERIMENTAL

In the following experiment, the protocol schematically depicted in FIG. 1 and described above is employed to produce sufficient high quality DNA template suitable for comprehensive high resolution microarray experiments. The following experiments show that the quality of the template generated according to the subject methods from degraded genomic samples is suitable for high-resolution CGH experiments.

Normal genomic DNAs are employed as genomic source to produce genomic template, as described above. These can consist of normal male, normal female, pooled male and female, or patient matched DNA derived from non-disease affected tissues. After restriction digestion, purification and quantification, 6 μg of the resultant genomic template is used as template in CGH labeling reactions reviewed below. In another experiment genomic DNAs from fresh frozen and paraffin embedded breast cancer tissues are used to generate template.

The resultant templates are purified with the Qiagen (Valencia, Calif.) Qiaquick PCR Cleanup kit. Cy3- or Cy5-dUTPs are incorporated into probes generated from the template, purified normal or tumor DNA respectively, using the BioPrime labeling kit (Invitrogen, Carlsbad, Calif.). Briefly, 6 μg genomic template is denatured in the presence of random octamers, then incubated with 3nmol Cy-labeled dUTP, unlabeled dNTPs and Klenow fragment for 2 hrs at 37° C. The labeling reaction is purified with Centricon YM-30 columns (Millipore Corp, Bedford, Md.). Cy3 and Cy5 samples are pooled, denatured and reannealed in the presence of 50 μg Cot-1 DNA, 20 μg yeast tRNA (Invitrogen, Carlsbad, Calif.) and 2.5 μl ×Agilent oligonucleotide microarray control target (Operon, Hayward, Calif.). Samples are then mixed with 2×Agilent deposition array buffer and hybridized to Human Catalogue arrays under coverslip overnight at 65° C. Hybridizations consist of the following combinations of DNA: a) non-amplified normal and non-amplified fresh frozen tumor, b) amplified normal and amplified fresh frozen tumor, c) non-amplified normal and non-amplified paraffin-embedded tumor, d) amplified normal and amplified paraffin-embedded tumor. Arrays are subsequently washed in buffer 1 (0.5×SSC, 0.001% Triton X-100) for 5 minutes at room temperature, then transferred to and washed in buffer 2 (0.1×SSC, 0.001% Triton X-100) for another 5 minutes at 37° C. The arrays are scanned on an Agilent microarray scanner and analyzed with Agilent feature extraction software.

The observed results demonstrate that the quality of the template generated according to the methods of the present invention from degraded genomic samples is suitable for high-resolution CGH experiments.

It is evident from the above results and discussion that this invention describes the development of protocols for preparing genomic templates from initially compromised genomic sources, such as archived samples. Advantages of the invention include the ability to produce accurate genomic templates from small amounts of degraded genomic sources, without having to reconstruct the genomic source material. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for producing a genomic template composition from a genomic source, said method comprising:
(a) contacting said genomic source with a precursor of a circular template nucleic acid, wherein said precursor comprises first and second domains that are at least partially complementary to substantially neighboring regions of a genomic domain of interest and said contacting occurs under conditions sufficient to ligate first and second domains via a genomic domain mediated ligation reaction to produce a ligated mixture; and
(b) subjecting said ligated mixture to template dependent primer extension reaction conditions to produce said genomic template composition.

2. The method according to claim 1, wherein said precursor is a linear nucleic acid comprising said first and second domains separated by a third spacer domain.

3. The method according to claim 2, wherein said third spacer domain comprises a restriction endonuclease site.

4. The method according to claim 2, wherein said genomic source is contacted with a plurality of different precursors.

5. The method according to claim 4, wherein all members of said plurality comprise the same third domain but different first and second domains.

6. The method according to claim 1, wherein said template dependent primer extension reaction conditions comprise amplification conditions.

7. The method according to claim 6, wherein said amplification conditions are isothermal.

8. The method according to claim 7, wherein said template dependent primer extension reaction conditions comprise rolling circle amplification (RCA) conditions.

9. The method according to claim 8, wherein said RCA conditions comprise contacting said second mixture with a highly processive polymerase.

10. The method according to claim 9, wherein said highly processive polymerase is a φ29-type polymerase.

11. The method according to claim 1, wherein said method further comprises preparing a collection of nucleic acid target molecules from said genomic template composition.

12. The method according to claim 11, wherein said method further comprises employing said collection of nucleic acid target molecules in a comparative genomic hybridization (CGH) assay.

13. The method according to claim 3, wherein said method comprises contacting said genomic template composition with an endonuclease that cleaves said restriction endonuclease site.

14. A method for comparing the copy number of at least one nucleic acid sequence in at least two genomic sources, said method comprising:
(a) preparing at least a-first genomic template from a first genomic source and a second genomic template from a second genomic source, wherein each of said first and second templates are prepared by:
(i) contacting a genomic source with a plurality of different target specific precursors of circular template nucleic acids, wherein each of said precursors comprises first and second domains that are at least partially complementary to substantially neighboring regions of a genomic domain of interest and said contacting occurs under conditions sufficient to ligate any proximal first and second domains via a target genomic domain mediated ligation reaction to produce a ligated mixture; and
(ii) subjecting said ligated mixture to rolling circle amplification reaction conditions to produce a genomic template composition;
to produce a first genomic template from said first genomic source and a second genomic template from said second genomic source
(b) preparing at least a first collection of nucleic acid target molecules from said first genomic template and a second collection of nucleic acid target molecules from said second genomic template;
(c) contacting said first and second collections of nucleic acid target molecules with one or more pluralities of oligonucleotide probe elements bound to a surface of a solid support, each probe element comprising a probe nucleic acid; and
(d) evaluating the binding of the first and second collections of nucleic acid target molecules to the same probe nucleic acid to compare the copy number of at least one nucleic acid sequence in said at least two genomic sources.

15. The method according to claim 14, wherein each of said collections of nucleic acid target molecules is labeled.

16. The method according to claim 14, wherein said contacting of said first and second collections of nucleic acid target molecules with one or more pluralities of oligonucleotide probe elements bound to a surface of a solid support occurs under stringent hybridization conditions.

17. The method according to claim 14, wherein the collections of nucleic acid target molecules are contacted with a single plurality of probe nucleic acids.

18. The method according to claim 17, wherein said collections of nucleic acid target molecules are distinguishably labeled.

19. The method according to claim 14, wherein each collection of nucleic acid target molecules is separately contacted with a plurality of probe nucleic acids.

20. The method according to claim 14, wherein said plurality of oligonucleotide probe elements bound to a surface of a solid support includes sequences representative of locations distributed across at least a portion of a genome.

21. A kit for use in comparing the relative copy number of at least one nucleic acid sequence in two or more genomes, said kit comprising:
(a) a plurality of oligonucleotide probe elements bound to a surface of a solid support, each probe element comprising a probe nucleic acid; and
(b) a precursor of a circular template comprising first and second domains that are at least partially complementary to substantially neighboring regions of a genomic domain of interest.

22. The kit according to claim 21, wherein said kit further includes a ligase.

23. The kit according to claim 22, wherein said kit further comprises at least one amplification reagent.

24. The kit according to claim 23, wherein said at least one amplification reagent is a highly processive polymerase.

25. The kit according to claim 21, wherein said kit further comprises first and second nucleic acid labeling reagents having distinguishable labels.

26. The kit according to claim 25, wherein said distinguishable labels are fluorescent distinguishable labels.

27. The kit according to claim 21, wherein said plurality of probe elements bound to a solid surface comprises an array.
